(19) United States
(12) Patent Application Publication
(10) Pub. No.: US 2021/0369663 A1
(43) Pub. Date: Dec. 2, 2021

(54) COMPOSITIONS AND METHODS USING A COMBINATION OF CURCUMIN AND AN OMEGA-3 FATTY ACID FOR CELLULAR ENERGY

(71) Applicant: SOCIETE DES PRODUITS NESTLE S.A., Vevey (CH)

(72) Inventors: Claire Boutry, Villars Burquin (CH); Denis Breuille, Lausanne (CH); Jerome Feige, Crissier (CH)

(21) Appl. No.: 17/291,055

(22) PCT Filed: Nov. 4, 2019
(86) PCT No.: PCT/EP2019/080068

§ 371 Date: May 4, 2021

Related U.S. Application Data
(60) Provisional application No. 62/755,833, filed on Nov. 5, 2018.

(51) Int. Cl. A61K 31/202; A23L 33/105; A23L 33/12; A61K 31/12

(52) U.S. Cl. CPC A61K 31/202; A61K 31/12; A23L 33/12; A23L 33/105

(57) ABSTRACT

Compositions may be used for a variety of therapeutic applications, including treating and/or preventing a disease or disorder related to reduced or inadequate mitochondrial activity, such as aging or stress, diabetes, obesity, and neurodegenerative diseases. The compositions can be administered to an older adult or an elderly individual. It can also be administered to a patient in ICU. The compositions contain a combination of curcumin and an omega-3 fatty acid. The compositions can be food products, nutritional supplements or nutraceutical. The compositions can also be used advantageously in generally healthy individuals to increase or maintain metabolic rate, decrease percent body fat, increase or maintain muscle mass, manage body weight, improve or maintain mental performance (including memory), improve or maintain muscle performance, improve or maintain mood, and manage stress.

### BACKGROUND

The present disclosure generally relates to compositions and methods that can treat or prevent a mitochondria-related disease or condition associated with altered mitochondrial function or a reduced mitochondrial density, for example by increasing antioxidant capacity, reducing oxidative stress, and/or enhancing mitochondrial function, in some embodiments in an older adult or an elderly individual.

Population aging has been a remarkable demographic event. As the growth of the older population has outpaced the total population due to increased longevity, the proportion of older persons relative to the rest of the population has increased considerably due to decreased fertility rates. For example, one in every twelve individuals was at least 60 years of age in 1950, and one in every ten was aged 60 years or older by the end of 2000. By the end of 2050, the number of persons worldwide that is 60 years or over is projected to be one in every five.

Aged or aging individuals frequently suffer some degree of cognitive impairment, including decline in cognitive function, that progresses with age, and age-related changes in brain morphology and cerebrovascular function are commonly observed. Cognitive decline has been consistently reported with aging across a range of cognitive domains including processing speed, attention, episodic memory, spatial ability and executive function. Brain imaging studies have revealed that these normal age-related cognitive declines are associated with decreases in both grey and white matter volume in the brain, with the fronto-striatal system most heavily compromised with aging. These decreases in cortical volume can be attributed to a number of detrimental cellular processes involved with normal aging, such as accumulation of damage by free radicals over time leading to oxidative damage, chronic low-grade inflammation, homocysteine accumulation (which when elevated are a risk factor for cognitive impairment and dementia), and decreased mitochondrial efficiency. In addition to direct cellular damage, the brain is also indirectly impaired by insults to micro-vascular structures. It is evident that the pathology of aging and also dementia involves a complexity of these interacting factors which are linked together. For example, mitochondrial dysfunction leads to increased oxidative stress, and oxidative stress can trigger inflammation and vascular insults.

Furthermore, cognitive decline is an early predictor or Alzheimer pathology and begins before the onset of dementia. In this context, the cognitive composite score represents a reliable means to assess the cognitive decline preceding dementia. Considerable evidence suggests that maintaining brain health and preventing cognitive decline with advancing age may prevent or delay development of dementia due to Alzheimer's disease and other aged related neuropathologies.

Nutrition, education, physical exercise and cognitive exercise have been recently demonstrated as possible intervention to prevent cognitive decline with aging. An abundance of clinical, epidemiological, and individual evidence is in favor of individual nutritional factors that reduce dementia risk and age-related neurodegeneration. However, formal trial testing of nutritional interventions has yielded mixed results (Schmitt et al., Nutrition Reviews 68: S2-S5 (2010).

Furthermore, stress (generally, an animal's reaction to change that requires a physical, mental, or emotional adjustment or response) can cause health problems for the animal. Prolonged, uninterrupted, unexpected, and unmanageable stresses are the most harmful types of stress.

There are known methods for affecting stress and the symptoms and conditions caused by stress. Drugs, such as those that reduce depression, can be used to affect stress and its associated symptoms and conditions. Anti-depressants such as Prozac®, Deroxat® and Zoloft® or anxiolytics such as Xanax®, Temesta®, Lexomil® and Valium® are often prescribed for treating stress and affecting and the symptoms and conditions caused by stress. These methods, however, are often accompanied by one or more adverse side effects. Meditation, relaxation, hypnosis, exercise, counseling, and nutrition, are known methods for affecting stress and the symptoms and conditions caused by stress.

Furthermore, the psychobiological features of stress may present as manifestations of oxidative stress, i.e., an imbalance between the production and manifestation of reactive oxygen species and the ability of a biological system readily to detoxify the reactive intermediates or to repair the resulting damage. Disturbances in the normal redox state of tissues can cause toxic effects through the production of peroxides and free radicals that damage all of the components of the cell, including proteins, lipids, and DNA. Some reactive oxidative species can even act as messengers through a phenomenon called “redox signaling.”

In humans, oxidative stress is involved in many diseases. Examples include atherosclerosis, Parkinson's disease, heart failure, myocardial infarction, Alzheimer's disease, schizophrenia, bipolar disorder, fragile X syndrome, and chronic fatigue syndrome.

One source of reactive oxygen under normal conditions in humans is the leakage of activated oxygen from mitochondria during oxidative phosphorylation. Other enzymes capable of producing superoxide (O2-) are xanthine oxidase, NADPH oxidases and cytochromes P450. Hydrogen peroxide, another strong oxidizing agent, is produced by a wide variety of enzymes including several oxidases. Reactive oxygen species play important roles in cell signaling, a process termed redox signaling. Thus, to maintain proper cellular homeostasis a balance must be struck between reactive oxygen production and consumption.

### SUMMARY

In view of the experimental data disclosed later herein, the present inventors believe that curcumin and an omega-3 fatty acid synergistically enhance the efficiency of mitochondria to produce energy.

Accordingly, in a general embodiment, the present disclosure provides a method of increasing antioxidant capacity, reducing oxidative stress, and/or enhancing mitochondrial function, the method comprising administering to an individual in need thereof an effective amount of a combination of curcumin and an omega-3 fatty acid.

In another embodiment, the present disclosure provides a method of treating, reducing an incidence of, and/or reducing a severity of a mitochondria-related disease or condition associated with altered mitochondrial function or a reduced mitochondrial density, the method comprising orally administering to an individual in need thereof an effective amount of a combination of curcumin and an omega-3 fatty acid.

In a further embodiment, it provides a method of delaying off-set of metabolic decline, maintaining muscle mass, decreasing oxidative stress, maintaining immune function and/or maintaining cognitive function in a healthy older adult, the method comprising orally administering to the healthy older adult an effective amount of a combination of curcumin and an omega-3 fatty acid.

It also relates to a method of enhancing metabolizing of reactive oxygen species, improving glucose control and/or improving muscle function in an individual with at least one of obesity or diabetes, the method comprising orally administering to the individual an effective amount of a combination of curcumin and an omega-3 fatty acid.

In another embodiment, the disclosure provides
i) a method of improving mitochondrial function in an individual, the method comprising orally administering to the individual an effective amount of a combination of curcumin and an omega-3 fatty acid.
ii) a method of increasing metabolic rate, the method comprising orally administering to an individual an effective amount of a combination of curcumin and an omega-3 fatty acid.
iii) A method of improving or maintaining cognitive function, the method comprising orally administering to an individual an effective amount of a combination of curcumin and an omega-3 fatty acid.
iv) A method of enhancing at least one of mental performance or muscle performance, the method comprising orally administering to an individual an effective amount of a combination of curcumin and an omega-3 fatty acid.
v) The method of claim 22, wherein the individual is elderly an older adult, an elderly or a patient in ICU.
vi) A method of weight management, the method comprising orally administering to an individual an effective amount of a combination of curcumin and an omega-3 fatty acid.
vii) A method of increasing or maintaining mitochondrial function, the method comprising administering to an individual an effective amount of a combination of curcumin and an omega-3 fatty acid.

In a further embodiment, the present disclosure relates to a composition in a unit dosage form comprising an amount of a combination of curcumin and an omega-3 fatty acid effective for at least one of (i) treating, reducing an incidence of, or reducing a severity of a mitochondria-related disease or condition associated with altered mitochondrial function or a reduced mitochondrial density, (ii) increasing metabolic rate, (iii) improving or maintaining cognitive function, (iv) enhancing mental performance, (v) enhancing muscle performance, (vi) managing weight, or (vii) increasing or maintaining mitochondrial function.

In another embodiment, it provides a kit comprising curcumin and an omega-3 fatty acid in one or more containers.

An advantage of one or more embodiments provided by the present disclosure is to boost healthy aging of cells.

Another advantage of one or more embodiments provided by the present disclosure is to help off-set slowing of the metabolism associated with aging.

And another advantage of one or more embodiments provided by the present disclosure is to help increase fatty acids metabolism.

Yet another advantage of one or more embodiments provided by the present disclosure is to help the body to metabolize fat and increase lean body mass.

An advantage of one or more embodiments provided by the present disclosure is to help maintain heart health.

Another advantage of one or more embodiments provided by the present disclosure is to help support healthy LDL-cholesterol and fatty acid levels in the blood.

And another advantage of one or more embodiments provided by the present disclosure is to help maintain healthy muscle mass.

Yet another advantage of one or more embodiments provided by the present disclosure is to help reduce oxidative stress on the body.

Additional features and advantages are described herein and will be apparent from the following Figures and Detailed Description.

### BRIEF DESCRIPTION OF DRAWINGS

FIGS. 1-3 are graphs of data from the experimental example disclosed herein.

FIG. 1: Effect of curcumin (CUR), n-3 fatty acid (OM3) and the combination of curcumin and n-3 FA (CUR+OM3) on mitochondrial complexes activity in old rats. 20 months-old rats were fed either a control diet or the diet supplemented with curcumin and optionally n-3 FA for 1 month. Results were expressed as mean±S.E.M.

FIG. 2: Effect of curcumin (CUR), n-3 fatty acid (OM3) and the combination of curcumin and n-3 FA (CUR+OM3) on citrate synthase activity in old rats. 20 months-old rats were fed either a control diet or the diet supplemented with curcumin and optionally n-3 FA for 1 month. Results were expressed as mean±S.E.M.

FIG. 3: Effect of curcumin (CUR), n-3 fatty acid (OM3) and the combination of curcumin and n-3 FA (CUR+OM3) on MFN2/DRP1 protein level ratio in old rats. 20 months-old rats were fed either a control diet or the diet supplemented with curcumin and optionally n-3 FA for 1 month. Results were expressed as mean±S.E.M.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. A composition “consisting essentially of” contains at least 50 wt. % of the referenced components, preferably at least 75 wt. % of the referenced components, more preferably at least 85 wt. % of the referenced components, most preferably at least 95 wt. % of the referenced components.

The term “and/or” used in the context of “X and/or Y” should be interpreted as “X,” or “Y,” or “X and Y.” Similarly, “at least one of X or Y” should be interpreted as “X,” or “Y,” or “X and Y.” For example, “at least one of mental performance or muscle performance” should be interpreted as “mental performance or muscle performance,” or “muscle performance,” or “both mental performance and muscle performance.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. A food product typically includes at least one of a protein, a lipid, a carbohydrate and optionally includes one or more vitamins and minerals. The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the term “isolated” means removed from one or more other compounds or components with which the compound may otherwise be found, for example as found in nature. For example, “isolated” preferably means that the identified compound is separated from at least a portion of the cellular material with which it is typically found in nature. In an embodiment, an isolated compound is pure, i.e., free from any other compound.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improved,” “increased,” “enhanced” and the like refer to the effects of the composition disclosed herein, namely a composition comprising a combination of curcumin and an omega-3 fatty acid, relative to a composition lacking curcumin and an omega-3 fatty acid but otherwise identical. As used herein, “promoting” refers to enhancing or inducing relative to the level before administration of the composition disclosed herein.

The term “unit dosage form,” as used herein, refers to physically discrete units suitable as unitary dosages for human and animal subjects, each unit containing a predetermined quantity of the composition disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In some embodiments, the unit dosage form can be a predetermined amount of the active compounds in a serving of a food product, a predetermined amount of powder in a sachet, a predetermined amount of the active compounds in a capsule or a tablet, or a predetermined amount of the active compounds in a predetermined volume of liquid, preferably a therapeutically or prophylactically effective amount or a predetermined portion of a therapeutically or prophylactically effective amount.

A “subject” or “individual” is a mammal, preferably a human. The term “elderly” in the context of a human means an age from birth of at least 60 years, preferably above 63 years, more preferably above 65 years, and most preferably above 70 years. The term “older adult” in the context of a human means an age from birth of at least 45 years, preferably above 50 years, more preferably above 55 years, and includes elderly individuals.

“As used herein, “frailty” is defined as a clinically recognizable state of increased vulnerability resulting from aging-associated decline in reserve and function across multiple physiologic systems such that the ability to cope with everyday or acute stressors is compromised. A pre-frail stage, in which one or two of these criteria are present, identifies a high risk of progressing to frailty.

“Overweight” is defined for a human as a body mass index (BMI) between 25 and 30 kg/m². “Obese” is defined for a human as a BMI of at least 30 kg/m², for example 30-39.9 kg/m². “Weight loss” is a reduction of the total body weight. Weight loss may, for example, refer to the loss of total body mass in an effort to improve one or more of health, fitness or appearance.

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women.

As used herein, the term “metabolic syndrome” refers to a combination of medical disorders that, when occurring together, increase the risk of developing cardiovascular disease and diabetes. It affects one in five people in the United States and prevalence increases with age. Some studies have shown the prevalence in the United States to be an estimated 25% of the population. In accordance with the International Diabetes Foundation consensus worldwide definition (2006), metabolic syndrome is central obesity plus any two of the following:

Raised triglycerides: >150 mg/dL (1.7 mmol/L), or specific treatment for this lipid abnormality;

Reduced HDL cholesterol: <40 mg/dL (1.03 mmol/L) in males, <50 mg/dL (1.29 mmol/L) in females, or specific treatment for this lipid abnormality;

Raised blood pressure: systolic BP>130 or diastolic BP>85 mm Hg, or treatment of previously diagnosed hypertension; and

Raised fasting plasma glucose: (FPG)>100 mg/dL (5.6 mmol/L), or previously diagnosed type 2 diabetes.

As used herein, “neurodegenerative disease” or “neurodegenerative disorder” refers to any condition involving progressive loss of functional neurons in the central nervous system. In an embodiment, the neurodegenerative disease is associated with age-related cell death. Non-limiting examples of neurodegenerative diseases include Alzheimer's disease, Parkinson's disease, Huntington's disease, amyotrophic lateral sclerosis (also known as ALS and as Lou Gehrig's disease), AIDS dementia complex, adrenoleukodystrophy, Alexander disease, Alper's disease, ataxia telangiectasia, Batten disease, bovine spongiform encephalopathy (BSE), Canavan disease, corticobasal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple sclerosis, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome. As used herein, “cognitive function” refers to any mental process that involves symbolic operations, e.g., perception, memory, attention, speech comprehension, speech generation, reading comprehension, creation of imagery, learning, and reasoning, preferably at least memory.

Methods for measuring cognitive function are well-known and can include, for example, individual or battery tests for any aspect of cognitive function. One such test is the Prudhoe Cognitive Function Test by Margallo-Lana et al. (2003) J. Intellect. Disability Res. 47:488-492. Another such test is the Mini Mental State Exam (MMSE), which is designed to assess orientation to time and place, registration, attention and calculation, recall, language use and comprehension, repetition, and complex commands. As used herein, a “cognitive disorder” refers to any condition that impairs cognitive function. Non-limiting examples of a cognitive disorder include delirium, dementia, learning disorder, attention deficit disorder (ADD), and attention deficit hyperactivity disorder (ADHD). A “stress-induced or stress-related cognitive dysfunction” refers to a disturbance in cognitive function that is induced or related to stress.

As used herein, a “mood disorder” (also known as an affective disorder) refers to a disturbance in emotional state, such as is set forth in the Diagnostic and Statistical Manual of Mental Disorders, published by the American Psychiatric Association. Non-limiting examples of mood disorders include major depression, postpartum depression, dysthymia, and bipolar disorder. A “stress-induced or stress-related mood disorder” refers to a disturbance in emotional state that is induced or related to stress. Such mood disorders are sometimes referred to as reactive mood disorders and are distinguished from other mood disorders, e.g., “organic” mood disorders that are due to a medical or physical condition rather than a psychiatric illness.

As used herein, an “anxiety disorder” refers to a dysfunctional state of fear and anxiety, e.g., fear and anxiety that is out of proportion to a stressful situation or the anticipation of a stressful situation. Non-limiting examples of anxiety disorders include generalized anxiety disorder, panic disorder, panic disorder with agoraphobia, agoraphobia, social anxiety disorder, obsessive-compulsive disorder, and post-traumatic stress disorder. A “stress-induced or stress-related anxiety disorder” refers to a dysfunctional state of fear and anxiety that is induced or related to stress. Such anxiety disorders are sometimes referred to as reactive anxiety disorders and are distinguished from other anxiety disorders, e.g., “organic” anxiety disorders that are due to a medical or physical condition rather than a psychiatric illness.

### EMBODIMENTS

The present disclosure provides compositions comprising a combination of curcumin and an omega-3 fatty acid. In a preferred embodiment, the fatty acid comprises essential polyunsaturated fatty acids, namely linoleic acid (C18:2n-3) or α-linolenic acid (C18:3n-3), or long-chain polyunsaturated fatty acids such as eicosapentaenoic acid (C20:5n-3), docosahexaenoic acid (C22:6n-3), or any combination thereof. More preferably, the omega-3 fatty acid is eicosapentaenoic acid.

The curcumin may be present in amount of about 0.01 mg to about 2.0 g per serving, preferably from about 0.1 mg to about 2.0 g per serving, even more preferably from about 10.0 mg to about 1.0 g per serving.

The omega-3 fatty acid can be at least about 10 wt. %, preferably at least about 15 wt. %, based on total lipid content. In a preferred embodiment, the daily amount of the omega-3 fatty acid is from about 500 mg to about 5 g omega-3 fatty acid per day, preferably from 500 mg to 2.5 g omega-3 fatty acid per day, more preferably about 1.5 g to about 2 g omega-3 fatty acid per day. The omega-3 fatty acid preferably comprises eicosapentaenoic acid.

Some embodiments of the compositions disclosed herein can comprise a plant extract that provides at least a portion of the curcumin, for example an extract of the Curcuma longa plant, for example the roots thereof. The plant extract can be enriched in curcumin, for example at least about 10 wt. % curcumin, preferably at least about 20 wt. % curcumin, more preferably at least about 30 wt. % curcumin, most preferably at least about 50 wt. % curcumin. Additionally or alternatively, a portion of the curcumin can be isolated curcumin.

In some embodiments of the compositions disclosed herein, at least a portion of the curcumin is highly bioavailable curcumin. Curcumin has poor absorption, biodistribution, metabolism, and bioavailability. Thus, continuous research on curcumin found some possible ways to overcome these problems. To increase the bioavailability, longer circulation, better permeability, and resistance to metabolic processes of curcumin several formulations have been prepared which include nanoparticles, liposomes, micelles, and phospholipid complexes.

In addition to the curcumin, one or more additional polyphenols can be included in the composition, for example flavonoids, e.g., isoflavones, anthocyanins, proanthocyanidins and anthocyanidins, flavans, flavonols, flavones and flavanones. Specific examples of bioflavonoids are catechins (catechin, epicatechin, gallocatechin, epigallocatechin, epicatechin gallate, epigallocatechin gallate), oleuropein, hesperidin and genistein.

Another anti-inflammatory compound or antioxidant may optionally be used in the composition. For example, additional antioxidants may be provided as food compositions that are rich in antioxidants or as extracts thereof. A food composition that is “rich in antioxidants” has an ORAC (oxygen radical absorbance capacity) rating of at least 100 per 100 g of the composition.

Without being bound by theory, it is believed that various types of stress result in stress injury to mitochondria, thereby reducing their ability to perform numerous functions essential to overall cell function. The methods disclosed herein can be useful for treating conditions involving stress injury to mitochondria, which injury may be manifest in any of a number of ways including, but not limited to, mitochondrial disease.

Mitochondrial diseases are the result of either inherited or spontaneous mutations in mitochondrial DNA or nuclear DNA which lead to altered functions of the proteins or RNA molecules that normally reside in mitochondria. Problems with mitochondrial function, however, may only affect certain tissues as a result of factors occurring during development and growth that are not yet fully understood. Even when tissue-specific isoforms of mitochondrial proteins are considered, it is difficult to explain the variable patterns of affected organ systems in the mitochondrial disease syndromes seen clinically.

Mitochondrial diseases result from failures of the mitochondria, specialized compartments present in every cell of the body except red blood cells. Mitochondria are responsible for creating more than 90% of the energy needed by the body to sustain life and support growth. When they fail, less and less energy is generated within the cell. Cell injury and even cell death follow. If this process is repeated throughout the body, whole systems begin to fail, and the life of the person in whom this is happening is severely compromised. Mitochondrial diseases primarily affect children, but adult onset is becoming more recognized.

Diseases of the mitochondria appear to cause the most damage to cells of the brain, heart, liver, skeletal muscles, kidney (e.g., kidney failure), and the endocrine and respiratory systems.

Many symptoms in mitochondrial disorders are non-specific. The symptoms may also show an episodic course, with periodic exacerbations. The episodic condition of migraine, as well as myalgia, gastrointestinal symptoms, tinnitus, depression, chronic fatigue, and diabetes, have been mentioned among the various manifestations of mitochondrial disorders in review papers on mitochondrial medicine. In patients with mitochondrial disorders, clinical symptomatology typically occurs at times of higher energy demand associated with physiological stressors, such as illness, fasting, over-exercise, and environmental temperature extremes. Furthermore, psychological stressors also frequently trigger symptomatology, presumably due to higher brain energy demands for which the patient is unable to match with sufficient ATP production.

Depending on which cells are affected, symptoms may include loss of motor control, muscle weakness and pain, gastro-intestinal disorders and swallowing difficulties, poor growth, cardiac disease, liver disease, diabetes, respiratory complications, seizures, visual/hearing problems (e.g., vision loss or hearing loss), lactic acidosis, developmental delays and susceptibility to infection.

Mitochondrial diseases include, without limitation, Alper's disease; Barth syndrome; beta-oxidation defects; carnitine deficiency; carnitine-acyl-carnitine deficiency; chronic progressive external ophthalmoplegia syndrome; co-enzyme Q10 deficiency; Complex I deficiency; Complex II deficiency; Complex III deficiency; Complex IV deficiency; Complex V deficiency; CPT I deficiency; CPT II deficiency; creatine deficiency syndrome; cytochrome c oxidase deficiency; glutaric aciduria type II; Kearns-Sayre syndrome; lactic acidosis; LCHAD (long-chain acyl-CoA dehydrogenase deficiency); Leber's hereditary optic neuropathy; Leigh disease; lethal infantile cardiomyopathy; Luft disease; MAD (medium-chain acyl-CoA dehydrogenase deficiency); mitochondrial cytopathy; mitochondrial DNA depletion; mitochondrial encephalomyopathy, lactic acidosis, and stroke-like symptoms; mitochondrial encephalopathy; mitochondrial myopathy; mitochondrial recessive ataxia syndrome; muscular dystrophies, myoclonic epilepsy and ragged-red fiber disease; myoneurogenic gastrointestinal encephalopathy; neuropathy, ataxia, retinitis pigmentosa, and ptosis; Pearson syndrome; POLG mutations; pyruvate carboxylase deficiency; pyruvate dehydrogenase deficiency; SCHAD (short-chain acyl-CoA dehydrogenase deficiency); and very long-chain acyl-CoA dehydrogenase deficiency.

Accordingly, an aspect of the present disclosure is a composition in a unit dosage form comprising a combination of curcumin and an omega-3 fatty acid in an amount effective for treatment or prevention of at least condition selected from the group consisting of stress (e.g., early-life stress and/or effects therefrom), obesity, reduced metabolic rate, metabolic syndrome, diabetes mellitus, hyperlipidemia, neurodegenerative disease, cognitive disorder, stress-induced or stress-related cognitive dysfunction, mood disorder (e.g., stress-induced or stress-related mood disorder), anxiety disorder (e.g., stress-induced or stress-related anxiety disorder) and age-related neuronal death or dysfunction (e.g., age-related neuronal death or dysfunction not attributable to a specific neurodegenerative disease), trauma, infection (e.g. in ICU) or cancer.

Another aspect of the present disclosure is a method of treating at least condition selected from the group consisting of stress (e.g., early-life stress and/or effects therefrom), obesity, reduced metabolic rate, metabolic syndrome, diabetes mellitus, cardiovascular disease, hyperlipidemia, neurodegenerative disease, cognitive disorder, stress-induced or stress-related cognitive dysfunction, mood disorder (e.g., stress-induced or stress-related mood disorder), anxiety disorder (e.g., stress-induced or stress-related anxiety disorder) and age-related neuronal death or dysfunction (e.g., age-related neuronal death or dysfunction not attributable to a specific neurodegenerative disease), trauma, infection (e.g. in ICU) or cancer in an individual having the at least one condition. The method comprises administering to the individual a composition comprising a therapeutically effective amount of a combination of curcumin and an omega-3 fatty acid.

A further aspect of the present disclosure is a method of preventing at least one condition selected from the group consisting of stress, obesity, reduced metabolic rate, metabolic syndrome, diabetes mellitus, cardiovascular disease, hyperlipidemia, neurodegenerative disease, cognitive disorder, stress-induced or stress-related cognitive dysfunction, mood disorder (e.g., stress-induced or stress-related mood disorder), anxiety disorder (e.g., stress-induced or stress-related anxiety disorder) and age-related neuronal death or dysfunction (e.g., age-related neuronal death or dysfunction not attributable to a specific neurodegenerative disease) trauma, infection (e.g. in ICU) or cancer. The method comprises administering to an individual at risk of the at least one condition a composition comprising a prophylactically effective amount of a combination of curcumin and an omega-3 fatty acid.

In an embodiment of these methods, the hyperlipidemia that is treated or prevented comprises hypertriglyceridemia. In an embodiment of these methods, the hyperlipidemia that is treated or prevented comprises elevated free fatty acids. In an embodiment of these methods, the age-related neuronal death or dysfunction that is treated or prevented is by administration of the composition to an older adult, such as an elderly individual.

The stress that is treated or prevented can be early-life stress, i.e., stress experienced while under the age of five years from birth. Early-life stress has been reported to have a significant detrimental effect on cognitive performance, including psychological parameters such as increased rates of or susceptibility to depression, anxiety, and abnormal risk-taking behavior. Increased rates of attention-deficit/hyperactivity disorder (ADHD), post-traumatic stress disorder (PTSD), and major depression have been reported in individuals having experienced early-life stress.

Another aspect of the present disclosure is a method of delaying off-set of metabolic decline, maintaining muscle mass, decreasing oxidative stress, maintaining immune function and/or maintaining cognitive function in a healthy older adult. The method comprises administering to the healthy older adult an effective amount of a combination of curcumin and an omega-3 fatty acid.

Another aspect of the present disclosure is a method of improving mitochondrial function in an individual, such as an older adult or an elderly individual. The method comprises administering to the individual an effective amount of a combination of curcumin and an omega-3 fatty acid.

Yet another aspect of the present disclosure is a method of enhancing metabolizing of reactive oxygen species, improving glucose control and/or improving muscle function in an individual with at least one of obesity or diabetes. The method comprises administering to the individual an effective amount of a combination of curcumin and an omega-3 fatty acid.

Another aspect of the present disclosure is a method of improving mitochondrial function (preferably to benefit at least one of metabolism or strength) in an individual, such as an older adult or an elderly individual. The method comprises administering to the individual an effective amount of a combination of curcumin and an omega-3 fatty acid.

Yet another aspect of the present disclosure is a composition comprising curcumin and optionally an omega-3 fatty acid in an amount effective for weight management. “Weight management” for an adult (e.g., at least eighteen years from birth) means that the individual has approximately the same body mass index (BMI) after one week of consumption of the composition, preferably after one month of consumption of the composition, more preferably after one year of consumption of the composition, relative to their BMI when consumption of the composition was initiated. “Weight management” for younger individuals means that the BMI is approximately the same percentile relative to an individual of a corresponding age after one week of consumption of the composition, preferably after one month of consumption of the composition, more preferably after one year of consumption of the composition, relative to their BMI percentile when consumption of the composition was initiated. In some embodiments, the individual undergoing weight management is an overweight individual preventing obesity.

In a related embodiment, method of weight management in an individual comprises administering to the individual a composition comprising an effective amount of a combination of curcumin and an omega-3 fatty acid.

Another aspect of the present disclosure is a composition in a unit dosage form comprising a combination of curcumin and an omega-3 fatty acid in an amount effective to increase at least one of muscle performance or mental performance (e.g., memory). In a related embodiment, a method of increasing at least one of muscle performance or mental performance (e.g., memory) in an individual comprises administering to the individual a composition comprising an effective amount of a combination of curcumin and an omega-3 fatty acid.

Further regarding muscle performance, the increased muscle performance may be one or more of improved muscle function, reduced decline in muscle function, improved muscle strength, improved muscle endurance and improved muscle recovery. The composition can improve physical endurance (e.g., ability to perform a physical task such as exercise, physical labor, sports activities), inhibit or retard physical fatigue, enhance blood oxygen levels, enhance energy in healthy individuals, enhance working capacity and endurance, reduce muscle fatigue, reduce stress, enhance function of cardiac muscle cells, improve sexual ability, increase muscle ATP levels, and/or reduce lactic acid in blood. “Endurance capacity” refers to the time to fatigue when exercising at a constant workload, generally at an intensity <80% VO₂max. In some embodiments, the composition is administered in an amount that increases mitochondrial activity, increases mitochondrial biogenesis, and/or increases mitochondrial mass. In some embodiments, the composition is administered in combination with an exercise and/or an exercise regimen to improve muscle performance and/or muscle endurance.

In some embodiments, the combination of curcumin and an omega-3 fatty acid is administered to an individual having impaired physical performance, impaired endurance capacity, and/or impaired muscle function. Improved muscle function can be particularly beneficial in elderly subjects with reduced muscle cell function as a result of an age-related condition. For example, a subject who may benefit from improved muscle cell function may experience a decline in muscle function which then leads to pre-frailty and frailty. Such subjects may not necessarily experience muscle wastage in addition to their decline in muscle function. Some subjects do experience both muscle wasting and a decline in muscle function. The combination of curcumin and an omega-3 fatty acid may enhance muscle performance in a subject who is frail or pre-frail.

Sports performance refers to the ability of an athlete's muscles to perform when participating in sports activities. Enhanced sports performance, strength, speed, and endurance are measured by an increase in muscular contraction strength, an increase in amplitude of muscle contraction, or a shortening of muscle reaction time between stimulation and contraction. “Athlete” refers to an individual who participates in sports at any level and who seeks to achieve an improved level of strength, speed, or endurance in their performance, such as, for example, body builders, bicyclists, long distance runners, and short distance runners. Enhanced sports performance is manifested by the ability to overcome muscle fatigue, ability to maintain activity for longer periods of time, and have a more effective workout.

The compositions and the methods disclosed herein can also be effective in the treatment of muscle-related pathological conditions, including myopathies; neuromuscular diseases, such as Duchenne muscular dystrophy; and/or cachexia associated with burns, bed rest, limb immobilization, or major thoracic, abdominal, and/or orthopedic surgery.

A further aspect of the present disclosure is a composition comprising a combination of curcumin and an omega-3 fatty acid an amount effective to increase or maintain at least one of mitochondrial function or metabolic rate. In a related embodiment, a method of increasing or maintaining at least one of mitochondrial function or metabolic rate in an individual comprises administering to the individual a composition comprising an effective amount of a combination of curcumin and an omega-3 fatty acid.

Yet another aspect of the present disclosure is a composition in a unit dosage form comprising a combination of curcumin and an omega-3 fatty acid in an amount effective to treat, prevent, or manage at least one of a mitochondria-related disease, a condition associated with an altered mitochondrial function, or a reduced mitochondrial density. In a related embodiment, a method of treating an individual having at least one of a mitochondria-related disease, a condition associated with an altered mitochondrial function, or a reduced mitochondrial density comprises administering to the individual a composition comprising an effective amount of a combination of curcumin and an omega-3 fatty acid. In another related embodiment, a method of preventing at least one of a mitochondria-related disease, a condition associated with an altered mitochondrial function, or a reduced mitochondrial density in an individual at risk thereof comprises administering to the individual a composition comprising an effective amount of a combination of curcumin and an omega-3 fatty acid.

Another aspect of the present disclosure is a composition in a unit dosage form comprising a combination of curcumin and an omega-3 fatty acid in an amount effective to improve or maintain cognitive function. In a related embodiment, a method of improving or maintaining cognitive function in an individual comprises administering to the individual a composition comprising a combination of curcumin and an omega-3 fatty acid.

In an embodiment, the individual does not have a cognitive disorder. For example, the composition can enhance cognitive function in a subject having normal cognitive function.

The compositions disclosed herein can also be used in the treatment of any of a variety of additional diseases and conditions in which defective or diminished mitochondrial activity participates in the pathophysiology of the disease or condition, or in which increased mitochondrial function will yield a desired beneficial effect. Non-limiting examples of such conditions include male infertility associated with diminished sperm motility, hearing loss, macular degeneration and other age-related and inherited eye disorders, and hearing loss (e.g., age-related hearing loss).

In each of the compositions and methods disclosed herein, the combination of curcumin and an omega-3 fatty acid can be administered in a composition that is preferably a food product, including food additives, food ingredients, functional foods, dietary supplements, medical foods, nutraceuticals, or food supplements.

In an embodiment, the composition further comprises a medium-chain triglyceride, for example one or more of caproic acid, caprylic acid, capric acid and lauric acid. In an embodiment, the composition further comprises a phospholipid, for example phosphatidylcholine.

In an embodiment, the composition further comprises a source of protein, preferably purified protein (i.e., isolated from the native food ingredient in which it was created). The protein content of the composition is preferably 20-99 wt. % of the composition, for example 20-90 wt. % of the composition, for example, 30-80 wt. % of the composition, for example 40-80 wt. % of the composition, for example 50-80 wt. %, for example 40-70 wt. % of the composition.

Non-limiting examples of suitable protein or sources thereof for use in the compositions include hydrolyzed, partially hydrolyzed or non-hydrolyzed proteins or protein sources. They may be derived from any known or otherwise suitable source such as milk (e.g., casein, whey), animal (e.g., meat, fish), cereal (e.g., rice, corn) or vegetable (e.g., soy, pea) sources. Combinations of sources or types of proteins may be used. Non-limiting examples of proteins or sources thereof include intact pea protein, intact pea protein isolates, intact pea protein concentrates, milk protein isolates, milk protein concentrates, casein protein isolates, casein protein concentrates, whey protein concentrates, whey protein isolates, sodium or calcium casemates, whole cow's milk, partially or completely defatted milk, yoghurt, soy protein isolates and soy protein concentrates, and combinations thereof. Combinations of sources or types of proteins may be used. Preferred proteins include pea protein, whey protein, soy protein and casein. Casein proteins may, for example, comprise sodium caseinate and calcium caseinate.

The source of protein may be provided by individual amino acids, polypeptides comprising amino acids, or mixtures thereof. For many muscle growth, muscle maintenance and/or muscle enhancement treatments, particular amino acids beneficial, for example L-arginine, L-glutamine, lysine and the branched-chain amino acids (i.e. leucine, isoleucine, and valine; in particular leucine and isoleucine). These particular amino acids may be provided as the source of protein or they may be additional to a main source of protein. Thus, the source of protein in the composition may include one or more branched-chain amino acids (leucine, isoleucine, and valine); one or both of L-arginine and L-glutamine; and lysine. In a preferred embodiment, the composition comprises whey protein and/or casein protein together with one or more individual amino acids, for example one or more of (or all of) leucine, isoleucine and L-arginine.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The compositions disclosed herein may be administered to the subject orally, enterally or parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

The present disclosure also provides a kit comprising curcumin and an omega-3 fatty acid in one or more containers. In some embodiments, one or more of these compounds can be isolated compounds.

In an embodiment of the kit, the curcumin and the omega-3 fatty acid can be provided together in one or more prepackaged unit dosage forms, for example in separate containers that each contain a dried powder such that each container contains one prepackaged unit dosage form.

In another embodiment, the kit can comprise a plurality of compositions for admixing together to form one or more of the compositions disclosed herein. For example, the kit can contain two or more dried powders in separate containers relative to each other, the separate powders each containing a portion of the final unit dosage form. As a non-limiting example of such an embodiment, the kit can contain one or more first containers that house the curcumin and can also contain one or more second containers that house the omega-3 fatty acid. The content of one of the first containers can be admixed with one of the second containers to form at least a portion of the unit dosage form of the composition.

### EXAMPLE

The following non-limiting example presents scientific data developing and supporting the concept of administering a combination of curcumin and an omega-3 fatty acid to increase cellular energy production and thereby increase of function of different tissues, such as muscle, which are reduced with age.

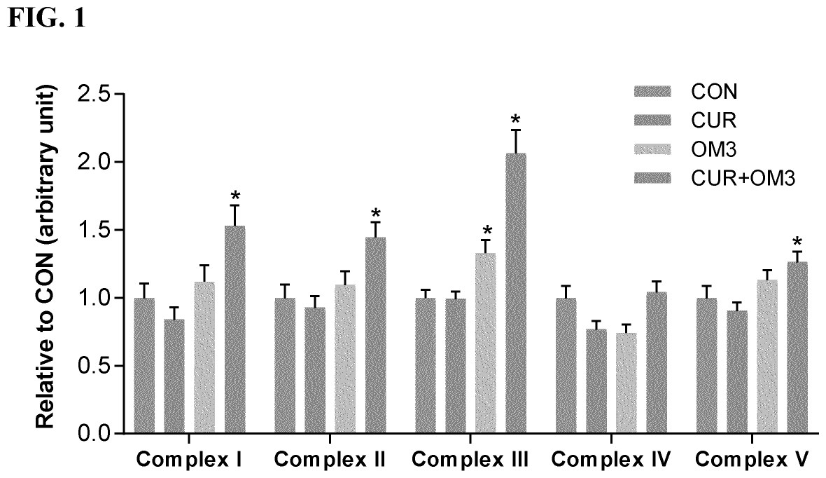

Old rats are a good model to assess the effect of nutritional intervention in age-related decline. In this model used by the present inventors, 20 months-old rats were fed for 1 month either with a normal diet or with the same diet supplemented with curcumin or n-3 fatty acid (n-3 FA) alone or in combination. Citrate synthase and mitochondrial respiratory complexes activities measured by colorimetry were synergistically enhanced in the curcumin and n-3 FA group, suggesting a better mitochondrial energy production. At the same time, there was a synergistic increase of MFN2/DRP1 expression ratio (mitochondria fusion/fission) measured by western blot in the same treatment group (FIGS. 1-3).

FIG. 1 shows the effect of curcumin (CUR), n-3 FA (OM3) and the combination of both (CUR+OM3) on mitochondrial complexes activity in old rats. 20 months-old rats were fed either a control diet or the same diet supplemented with curcumin, n-3 FA and the combination of both for 1 month. Results were expressed as mean±S.E.M. CON: control diet. CUR: control diet with curcumin. OM3: control diet with n-3 fatty acids. CUR+OM3: control diet with curcumin and n-3 fatty acids.

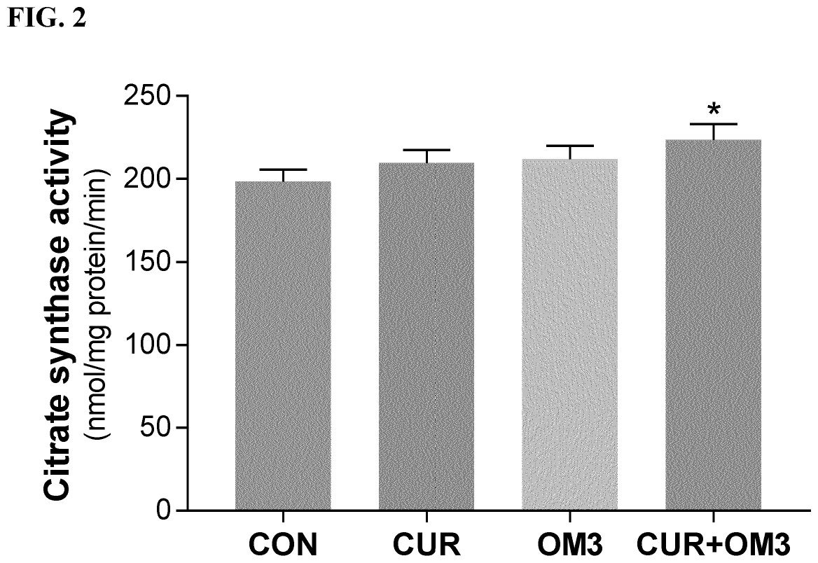

FIG. 2 shows the effect of curcumin (CUR), n-3 FA (OM3) and the combination of both (CUR+OM3) on citrate synthase activity in old rats. 20 months-old rats were fed either a control diet or the same diet supplemented with curcumin, n-3 FA and the combination of both for 1 month. Results were expressed as mean±S.E.M. CON: control diet. CUR: control diet with curcumin. OM3: control diet with n-3 fatty acids. CUR+OM3: control diet with curcumin and n-3 fatty acids.

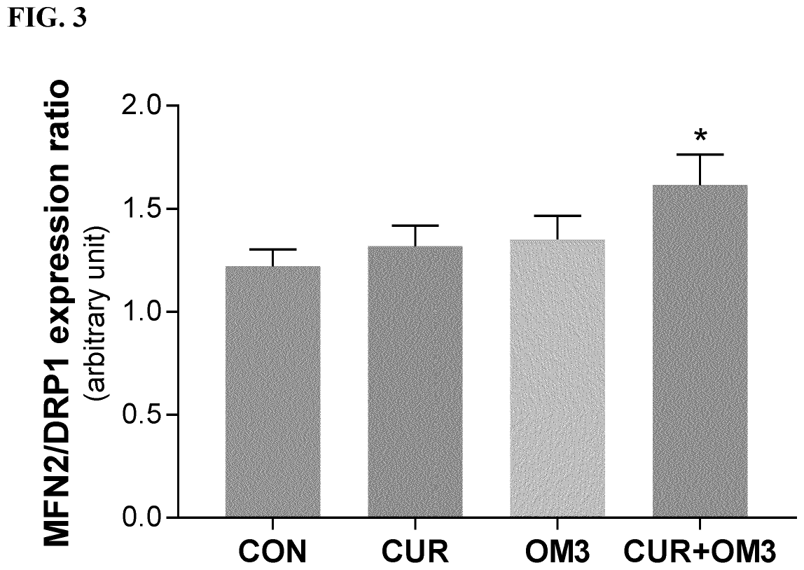

FIG. 3 shows the effect of curcumin (CUR), n-3 FA (OM3) and the combination of both (CUR+OM3) on MFN2/DRP1 expression ratio in old rats. 20 months-old rats were fed either a control diet or the same diet supplemented with curcumin, n-3 FA and the combination of both for 1 month. Results were expressed as mean±S.E.M. CON: control diet. CUR: control diet with curcumin. OM3: control diet with n-3 fatty acids. CUR+OM3: control diet with curcumin and n-3 fatty acids.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of increasing antioxidant capacity, reducing oxidative stress, and/or enhancing mitochondrial function, the method comprising administering to an individual in need thereof an effective amount of a combination of curcumin and an omega-3 fatty acid.

2. The method of claim 1, wherein the combination is administered in an amount providing from about 500 mg to about 5 g of the omega-3 fatty acid per day.

3. (canceled)

4. The method of claim 1, wherein the combination is administered orally.

5. The method of claim 1, wherein the combination is administered in a food product that comprises a plant extract that provides at least a portion of the curcumin.

6. The method of claim 1, wherein the individual is selected from the group consisting of an older adult and an elderly individual.

7. The method of claim 1, wherein the individual is a patient in ICU.

8. A method of treating, reducing an incidence of, and/or reducing a severity of a mitochondria-related disease or condition associated with altered mitochondrial function or a reduced mitochondrial density, the method comprising orally administering to an individual in need thereof an effective amount of a combination of curcumin and an omega-3 fatty acid.

9. The method of claim 8, wherein the mitochondria-related disease or condition is selected from the group consisting of stress, obesity, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, musculoskeletal disorder, frailty, pre-frailty, chronic kidney disease, kidney failure, trauma, infection, cancer, hearing loss, macular degeneration, myopathies and dystrophies, and combinations thereof.

10. The method of claim 8, wherein the mitochondria-related disease or condition comprises early-life stress and/or effects therefrom.

11. The method of claim 8, wherein the mitochondria-related disease or condition comprises hyperlipidemia comprising at least one of hypertriglyceridemia or elevated free fatty acids.

12. The method of claim 8, wherein the mitochondria-related disease or condition comprises at least one of stress-induced or stress-related mood disorder or stress-induced or stress-related anxiety disorder.

13. The method of claim 8, wherein the mitochondria-related disease or condition comprises age-related neuronal death or dysfunction not attributable to a specific neurodegenerative disease.

14-19. (canceled)

20. A method of improving or maintaining cognitive function, the method comprising orally administering to an individual an effective amount of a combination of curcumin and an omega-3 fatty acid.

21. The method of claim 20, wherein the cognitive function is selected from the group consisting of perception, memory, attention, speech comprehension, speech generation, reading comprehension, creation of imagery, learning, reasoning, and combinations thereof.

22. The method of claim 20, wherein the individual does not have a cognitive disorder.

23. The method of claim 20, wherein the individual is an older adult, an elderly or a patient in ICU.

24-34. (canceled)
